(19) United States
(12) Patent Application Publication
(10) Pub. No.: US 2026/0283694 A1
(43) Pub. Date: Sep. 24, 2026

(54) DYNAMIC LASER POWER MANAGEMENT

(71) Applicant: Lumenis Ltd., Yokneam llit (IL)

(72) Inventor: Hernan Altman, Kiryat Tiv'on (IL)

(73) Assignee: Lumenis Ltd., Yokneam llit (IL)

(21) Appl. No.: 19/572,222

(22) Filed: Mar. 19, 2026

Related U.S. Application Data
(60) Provisional application No. 63/774,456, filed on Mar. 19, 2025.

(51) Int. Cl. A61B 18/26; A61B 18/00

(52) U.S. Cl. CPC A61B 18/26; A61B 2018/00642; A61B 2018/00714; A61B 2018/00744; A61B 2018/00761; A61B 2018/00791; A61B 2218/002

(57) ABSTRACT

A medical laser system and associated methods for dynamically managing laser power during surgical procedures are disclosed. The system includes a laser console having a laser source, an optical coupler for delivering laser energy through an optical fiber, and a controller with a processor and memory. The controller receives operating parameters including laser on-time and off-time, and determines a temperature and/or thermal dose as a function of time based on the parameters. The thermal dose may be derived using models, lookup tables, or machine learning. The system automatically adjusts laser operating parameters, such as pulse energy or frequency, in response to the determined thermal dose to improve treatment efficiency while reducing risk of tissue damage.

### CROSS-REFERENCE TO RELATED APPLICATIONS

The application claims the benefit of U.S. Provisional Patent Application Serial No. 63/774,456 filed on Mar. 19, 2025, the disclosure of which is incorporated herein by reference.

### BACKGROUND

Medical lasers are used in a variety of procedures. Among several of the procedures, laser energy is directed towards a target using a fiber as a conduit for the laser energy. One such procedure, to address renal calculi (e.g. kidney stones) is ureteral endoscopy, or lithotripsy. An endoscopic probe, with a camera or other sensor, is inserted into the patient’s urinary tract to locate the calculi for removal. In endoscopic lithotripsy, the probe also includes a working channel in which an optical fiber is inserted. The optical fiber conducts a laser beam to disintegrate the calculi as they are found.

An ideal lithotripsy procedure is fast, precise, and thorough. The medical practitioner minimizes the time that the endoscope is inserted, directs all the discharged laser energy into target calculi, and minimizes risk of thermal damage. However, even with advancements in technology, modern devices do not have a method to accurately measure temperature within the treatment areas or accurately estimate the amount of thermal energy delivered to the treatment area, and to automatically adjust laser frequency to provide maximum treatment efficiency without damaging the surrounding tissue.

### BRIEF SUMMARY

This disclosure provides design, material, manufacturing method, and use alternatives for medical devices. An example medical device includes a medical laser console. The medical laser console can comprise a laser source configured to generate laser light; an optical coupler to couple the laser light to an optical fiber; and a controller, the controller comprising at least a processor and a memory, the memory comprising instructions, when executed by the processor, cause the controller to: receive a plurality of operating parameters, including: a first time generated by a laser system, the first time corresponding to a time that a laser is on, and a second time generated by the laser system, the second time corresponding to a time that the laser is off; determine a predicted temperature based on the plurality of operating parameters; and determine, during a time period, a thermal dose corresponding to the operating parameters and the predicted temperature, wherein the thermal dose is represented as a function of time.

With further embodiments of the medical laser console, the plurality of operating parameters further includes a temperature.

With further embodiments, the medical laser console can comprise an irrigation system.

With further embodiments of the medical laser console, the temperature is a fluid temperature from the irrigation system.

With further embodiments of the medical laser console, the plurality of operating parameters further includes a fluid flow rate and/or suction rate from the irrigation system, fiber size, scope size, sheath size, etc.

With further embodiments of the medical laser console, the instructions, when executed by the processor cause the controller to identify a reference in a lookup table corresponding to a temperature value and/or a thermal dose, wherein the lookup table is stored in a memory coupled to the processor, and wherein the lookup table correlates the plurality of operating parameters to the thermal dose; and determine the thermal does based on the reference.

With further embodiments of the medical laser console, the instructions, when executed by the processor cause the controller to determine whether the thermal dose is greater than or equal to a threshold thermal dose; and send a control signal to the laser source to cause the laser source to stop generating the laser light responsive to a determination that the thermal dose is greater than or equal to the threshold thermal dose.

With further embodiments of the medical laser console, the instructions, when executed by the processor cause the controller to compare the temperature to a predetermined temperature or compare the thermal dose to a predetermined thermal dose; and determine if the thermal dose is safe or unsafe, wherein as the thermal does increases above a predetermined threshold, the thermal dose becomes unsafe, and wherein as the thermal dose decreases below a predetermined threshold, the thermal dose becomes safe.

With further embodiments of the medical laser console, the instructions, when executed by the processor, cause the controller to decrease a frequency of lasing when the thermal dose is unsafe.

With further embodiments of the medical laser console, the instructions, when executed by the processor, cause the controller to increase a parameter (e.g., a frequency, a pulse energy, or both) of lasing when the temperature and/or thermal dose is safe.

With further embodiments of the medical laser console, the instructions, when executed by the processor, cause the controller further comprising determining at the processor, a distance of one or more of a plurality of objects from a distal end of the optical fiber.

With further embodiments of the medical laser console, the instructions, when executed by the processor, cause the controller to compare the distance to a predetermined threshold distance; and determine if the one or more of a plurality of objects is in range, or out of range.

With further embodiments of the medical laser console, the instructions, when executed by the processor, cause the controller to increase the pulse energy and/or frequency of lasing when the thermal dose is safe, and the one or more of a plurality of objects is in range.

With further embodiments, the medical laser console can comprise an endoscope configured to receive and support the optical fiber, the endoscope including a temperature sensor disposed on a distal end of the endoscope, the temperature sensor configured to determine a local temperature.

With further embodiments of the medical laser console, the laser console comprises user interface elements which may facilitate display, execution, interaction, manipulation, or operation of program components through textual or graphical facilities.

In some embodiments, the disclosure can be implemented as a method of determining a thermal dose. The method can comprise receiving, at a controller for a surgical laser system, a plurality of operating parameters, including a first time generated by a laser system, the first time corresponding to a time that a laser is on; a second time generated by the laser system, the second time corresponding to a time that the laser is off; determining, using a simulation model, a temperature prediction based on the plurality of operating parameters; and determining, during a time period, a thermal dose corresponding to the temperature prediction as a function of time.

With further embodiments, the method can comprise comparing the thermal dose to a predetermined temperature threshold; and determining if the thermal dose is safe or unsafe.

With further embodiments of the method, as the thermal does increases above a predetermined threshold, the thermal dose becomes unsafe, and wherein as the thermal dose decreases below a predetermined threshold, the thermal dose becomes safe. In some embodiments, the thermal dose is not considered safe until a predetermined time has passed after the thermal dose has decreased below the threshold.

With further embodiments, the method can comprise decreasing a pulse energy and/or frequency of lasing when the temperature and/or thermal dose is unsafe and increase a pulse energy and/or frequency of lasing when the temperature and/or thermal dose is safe.

In some embodiments, the disclosure can be implemented as at least one non-transitory computer-readable storage medium comprising instructions executable by a processor of a surgical laser console. The instructions, when executed, cause the surgical laser console to receive a plurality of operating parameters, including: a first time generated by a laser system, the first time corresponding to a time that a laser source of the surgical laser console is on; a second time generated by the laser system, the second time corresponding to a time that the laser source is off; and determine, using a model, a predicted temperature based on the plurality of operating parameters.

With further embodiments of the at least one non-transitory computer-readable storage medium, the predicted temperature is determined based on a machine learning model.

With further embodiments of the at least one non-transitory computer-readable storage medium, the predicted temperature is derived by an algorithm.

With further embodiments of the at least one non-transitory computer-readable storage medium, the instructions when executed by the processor further cause the surgical laser system to: determine whether the temperature and/or thermal dose is greater than or equal to a threshold thermal dose; and send a control signal to the laser source to cause the laser source to stop generating the laser light responsive to a determination that the thermal dose is greater than or equal to the threshold thermal dose.

The above summary of some embodiments, aspects, and/or examples is not intended to describe each embodiment or every implementation of the present disclosure. The figures and the detailed description which follows more particularly exemplify these embodiments.

### BRIEF DESCRIPTION OF THE DRAWINGS

To easily identify the discussion of any element or act, the most significant digit or digits in a reference number refer to the figure number in which that element is first introduced.

FIG. 1A illustrates a computer system in communication with a laser system. The laser system emitting laser light through an optical fiber and receiving reflected laser light back.

FIG. 1B illustrates an optic fiber emitting laser light onto a target and receiving reflected light back.

FIG. 2 illustrates an example lasing system.

FIG. 3 illustrates, in block form, a method for determining a thermal dose;

FIG. 4 illustrates in block form, comparing thermal dose to a threshold;

FIG. 5 illustrates a graph in accordance with one embodiment;

FIG. 6 illustrates computer executable instructions;

FIG. 7 illustrates a block diagram of a computing environment.

### DETAILED DESCRIPTION

For the following defined terms, these definitions shall be applied, unless a different definition is given in the claims or elsewhere in this specification.

All numeric values are herein assumed to be modified by the term “about,” whether explicitly indicated. The term “about”, in the context of numeric values, generally refers to a range of numbers that one of skill in the art would consider equivalent to the recited value (e.g., having the same function or result). In many instances, the term “about” may include numbers that are rounded to the nearest significant figure. Other uses of the term “about” (e.g., in a context other than numeric values) may be assumed to have their ordinary and customary definition(s), as understood from and consistent with the context of the specification, unless otherwise specified.

The recitation of numerical ranges by endpoints includes all numbers within that range, including the endpoints (e.g., 1 to 5 includes 1, 1.5, 2, 2.75, 3, 3.80, 4, and 5). Although some suitable dimensions, ranges, and/or values pertaining to various components, features and/or specifications are disclosed, one of skill in the art, incited by the present disclosure, would understand desired dimensions, ranges, and/or values may deviate from those expressly disclosed.

As used in this specification and the appended claims, the singular forms “a”, “an”, and “the” include plural referents unless the content clearly dictates otherwise. As used in this specification and the appended claims, the term “or” is generally employed in its sense including “and/or” unless the content clearly dictates otherwise. It is to be noted that to facilitate understanding, certain features of the disclosure may be described in the singular, even though those features may be plural or recurring within the disclosed embodiment(s). Each instance of the features may include and/or be encompassed by the singular disclosure(s), unless expressly stated to the contrary. For simplicity and clarity purposes, not all elements of the disclosure are necessarily shown in each figure or discussed in detail below. However, it will be understood that the following discussion may apply equally to any and/or all the components for which there are more than one, unless explicitly stated to the contrary. Additionally, not all instances of some elements or features may be shown in each figure for clarity.

Relative terms such as “proximal”, “distal”, “advance”, “withdraw”, variants thereof, and the like, may be generally considered with respect to the positioning, direction, and/or operation of various elements relative to a user/operator/manipulator of the device, wherein “proximal” and “withdraw” indicate or refer to closer to or toward the user and “distal” and “advance” indicate or refer to farther from or away from the user. In some instances, the terms “proximal” and “distal” may be arbitrarily assigned to facilitate understanding of the disclosure, and such instances will be readily apparent to the skilled artisan. It is noted that references in the specification to “an embodiment”, “some embodiments”, “other embodiments”, etc., indicate that the embodiment(s) described may include a particular feature, structure, or characteristic, but every embodiment may not necessarily include the feature, structure, or characteristic. Moreover, such phrases are not necessarily referring to the same embodiment. Further, when a particular feature, structure, or characteristic is described in connection with an embodiment, it would be within the knowledge of one skilled in the art to affect the feature, structure, or characteristic in connection with other embodiments, whether explicitly described or not, unless clearly stated to the contrary. That is, the various individual elements described below, even if not explicitly shown in a particular combination, are nevertheless contemplated as being combinable or arrangeable with each other to form other additional embodiments or to complement and/or enrich the described embodiment(s), as would be understood by one of ordinary skill in the art.

To clarify, certain identifying numerical nomenclature (e.g., first, second, third, fourth, etc.) may be used throughout the description and/or claims to name and/or differentiate between various described and/or claimed features. It is to be understood that the numerical nomenclature is not intended to be limiting and is exemplary only. In some embodiments, alterations of and deviations from previously used numerical nomenclature may be made in the interest of brevity and clarity. That is, a feature identified as a “first” element may later be referred to as a “second” element, a “third” element, etc. or may be omitted entirely, and/or a different feature may be referred to as the “first” element. The meaning and/or designation in each instance will be apparent to the skilled practitioner.

The following description should be read with reference to the drawings, which are not necessarily to scale, wherein similar elements in different drawings are numbered the same. The detailed description and drawings are intended to illustrate but not limit the disclosure. Those skilled in the art will recognize that the various elements described and/or shown may be arranged in various combinations and configurations without departing from the scope of the disclosure. The detailed description and drawings illustrate example embodiments of the disclosure. However, in the interest of clarity and ease of understanding, while every feature and/or element may not be shown in each drawing, the feature(s) and/or element(s) may be understood to be present regardless, unless otherwise specified.

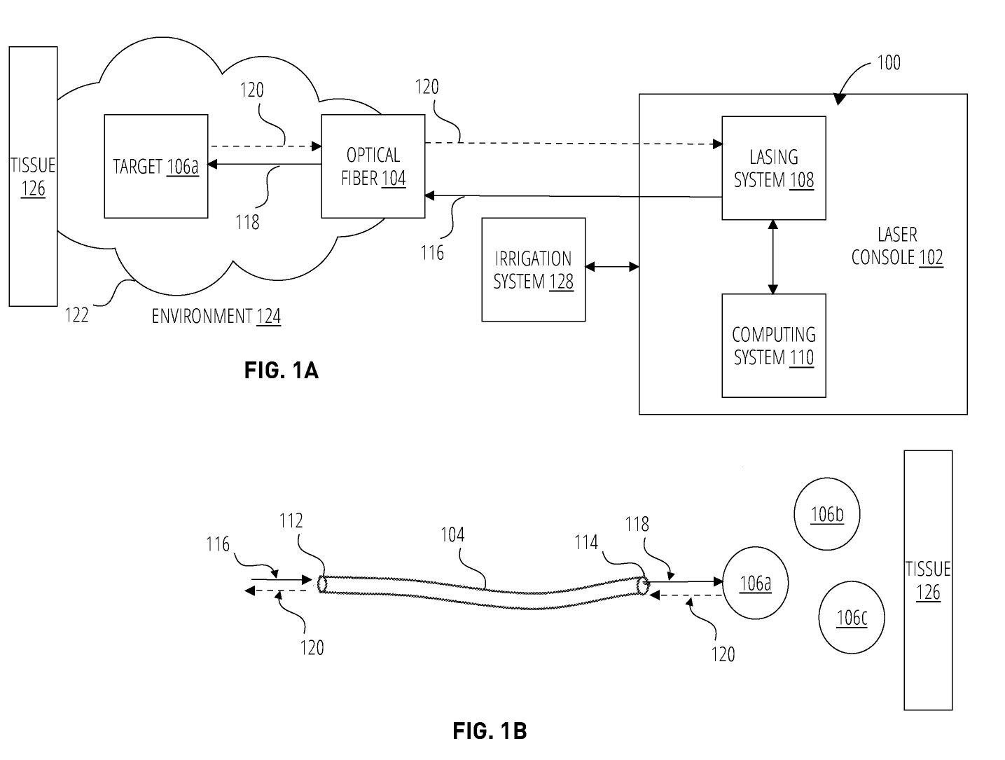

FIGS. 1A and 1B show an exemplary lithotripsy procedure system 100 for determining a temperature and/or thermal dose emitted from the fiber during a lithotripsy procedure in accordance with some embodiments of the present disclosure. It is noted that many example embodiemtns discuss determining the thermal dose. However, in some examples, merely the temperature can be determined. In such cases, the temperature can be used like the thermal dose to “close the loop” with the laser console and adjust lasing parameters as described herein.

In some embodiments, the exemplary lithotripsy procedure system 100 comprises a laser console 102 and an optical fiber 104 configured to emit laser light (e.g., emitted laser light 118) towards a target 106a. The laser console 102 can include a lasing system 108, computing system 110, and irrigation system 128.

In some embodiments, the target 106a may be a tissue, a stone, a tumor, a cyst, and the like, within a subject, which is to be treated, ablated, or destroyed. In some embodiments, the subject may be a human being or an animal. During operation, the optical fiber 104 is coupled to the laser console 102 and inserted an environment 124 of the target (e.g., via a ureteroscope, or the like) and placed proximate to a target 106a where laser energy can be generated by the laser console 102 and directed towards the target 106a via the optical fiber 104. When a target is not within the “aim” or longitudinal axis of the optical fiber 104, the laser energy may reach a tissue 126. Additionally, the environment 124 may be a liquid environment. In some embodiments, fluid can be supplied to the environment 124 by the irrigation system 128. The irrigation system 128 may be configured to determine and communicate fluid flow rate and fluid temperature information to the computing system 110.

The optical fiber is configured to be inserted within a working channel of an endoscope, for example a ureteroscope. The endoscope may include a variety of sensors and/or imaging devices, for example temperature sensors and cameras. The sensors may be configured to determine and communicate temperature, pressure, size, location, and other information to the computing system 110.

As depicted more fully in FIG. 1B, the optical fiber 104 comprises a proximal end 112 and a distal end 114. The proximal end 112 is the end of the optical fiber 104 coupled to the laser console 102 and through which light beams enter while the distal end 114 is the end of the optical fiber 104 through which light beams are emitted and via which light beams can be directed onto the target 106a. For example, FIG. 1B depicts laser light 116 entering the optical fiber 104 at the proximal end 112 and propagating through length of the optical fiber 104. A portion of laser light 116 exits the distal end 114 of the optical fiber 104 as emitted laser light 118 and is directed towards the target to be incident on the target 106a or the tissue 126. Furthermore, a portion of laser light 116 is reflected up the fiber by the target 106a or tissue 126 as reflected laser light 120. Furthermore, another portion of laser light 116 is reflected up the fiber by the distal end 114 of the optical fiber 104. That is, due to the interface between the distal end 114 and the environment 124 some laser light 116 will be reflected up the optical fiber 104 by the distal end 114. This is described in greater detail below.

As introduced above, often the environment 124 of the target will be a liquid environment (e.g., the bladder, kidney, or the like).

Laser light 116 can be generated by lasing system 108. Lasing system 108 may include, but is not limited to, solid-state lasers, gas lasers, diode lasers, and fiber lasers. As an illustrative example, lasing system 108 can be configured to generate laser light 116 using a Holmium-based lasing medium or a Thulium-based lasing medium. Lasing system 108 can comprise optical components which may include, but are not limited to, a lasing medium, pump lights, polarizers, beam splitters, beam combiners, light detector, wavelength division multiplexers, collimators, circulators, lenses, or other such optical components arranged in various combinations to provide laser light 116.

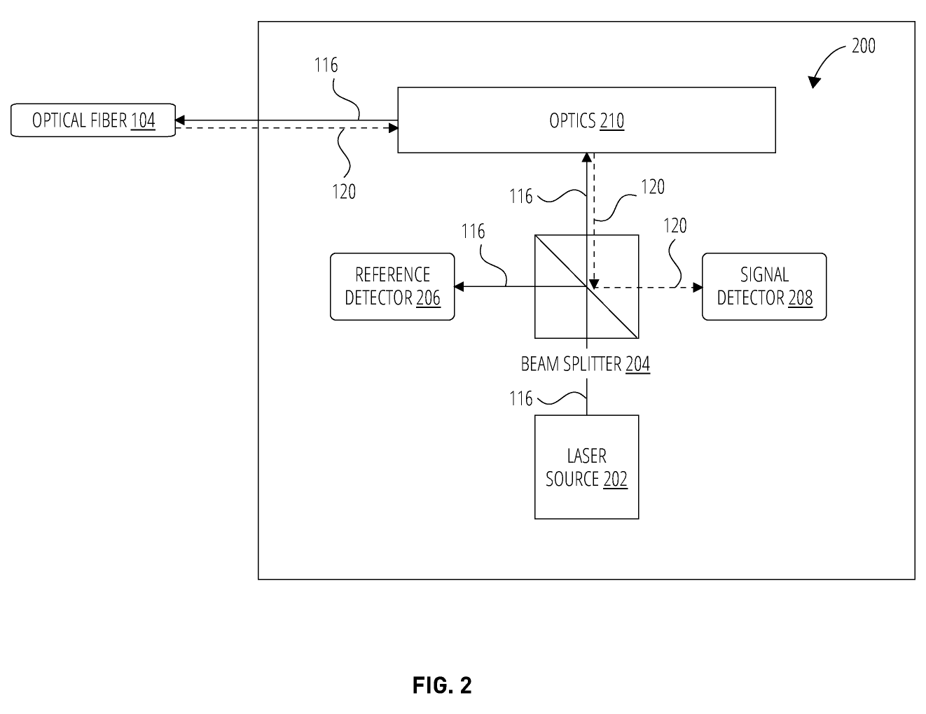

FIG. 2 illustrates an example lasing system 200. Lasing system 200 can include a laser source 202, a beam splitter 204, a reference detector 206, a signal detector 208, and optics 210. As described above, laser source 202 can be arranged to generate laser light 116 via several different lasing mechanisms, such as, for example, using a Holmium lasing medium, using a Thulium lasing medium, or the like.

Laser light 116 can be directed to beam splitter 204, which splits the laser light 116 to direct a portion of laser light 116 to optics 210 and a portion of laser light 116 to reference detector 206. Beam splitter 204 may include any of a variety of optical components used to split incident light at a designated ratio into two separate beams. Further, beam splitter 204 may be arranged to manipulate light to be incident at a desired angle of incidence (AOI). Therefore, in many embodiments, beam splitter 204 can be primarily configured with two parameters, a ratio of separation and an AOI. The ratio of separation comprises the ratio of reflection to transmission (reflection/transmission (R/T) ratio) of the beam splitter 204. Accordingly, as used herein, if the ratio of separation for a beam splitter 204 is indicated as 1:99, it means that the beam splitter 204 splits the incident light beams in a R/T ratio of 1:99. In other words, the beam splitter 204 splits the incident light beams by changing the incident light by reflecting 1 percent and transmitting the other 99 percent. Further, as an example, if the AOI for the beam splitter 204 is indicated as 45 degrees, it means that the beam splitter 204 ensures that the light beams would be incident at an angle of 45 degrees. Beam splitter 204 may include, but are not limited to, polarizing beam splitters and non-polarizing beam splitters. Polarizing beam splitters may split incident light based on the S-polarization component and P-polarization component, such as, for example by reflecting the S-polarized component of light and transmitting the P-polarized component of light (or vice-versa). In some embodiments, non-polarizing beam splitters may split incident light beams based on a specific R/T ratio while maintaining the original polarization state of the incident light beams. Optics 210 can comprise any of a variety of optical component arranged to condition and direct laser light 116 from beam splitter 204 to optical fiber 104 and direct reflected laser light 120 from optical fiber 104 to beam splitter 204. Optics 210 can include polarizers, beam combiners, collimators, circulators, lenses, etc.

From optics 210, reflected laser light 120 is directed to beam splitter 204, which reflects reflected laser light 120 to signal detector 208. Reference detector 206 and signal detector 208 can be any of a variety of light detectors. In general, such light detectors may include devices that detect and/or measure characteristics of light beams and encode the detected and/or measured characteristics in electrical signals. For example, light detectors may detect the specific type of light beams (as preconfigured), and convert the light energy associated with the detected light beams into electrical signals. These electrical signals can be communicated to a computing device (e.g., computing system 110, or the like) to determine the power of emitted laser light 118 as described herein. In general, the computing system 110 can include circuitry arranged to determine a distance between targets and/or tissue and the distal end 114 of the optical fiber 104, and to determine when a target is passing through laser light 116.

During a lithotripsy procedure, the lasing system 200 undergoes a duty cycle consisting of “on” periods where the laser source 202 is emitting laser light 116 at a frequency e.g. a predetermined repetition rate, and “off” periods where the laser source 202 is not emitting laser light 116. The duty cycle may be predetermined and controlled by the laser console 102 or may be manually adjusted throughout the procedure (e.g. duty cycle gating with a foot pedal). The lasing system 200 can be configured to communicate duty cycle information, for example length of on and off periods, to the computing device (e.g. Computing system 110). Time information can be provided to the computing device by the lasing system 200. In some embodiments, time information may be input as a predetermined duty cycle. The lasing system 200 can also be configured to communicate frequency information to the computing device.

The optics system 210 in conjunction with the circuitry can be used to determine if one of the targets 306, 308, 310 and 312 is an optimal range from the distal end 114 of the optical fiber 104. Additionally, the circuitry and optics system 210 may be used to determine when a stone is in the path of the laser light 116 based on reflected laser light 120 and the electrical signals converted by the light detectors. In some embodiments, emitted laser light 116 may cause a bubble 122 to form within the liquid environment 124. The circuitry can be configured to determine a size of the bubble 122 based on reflected laser light received by the light detectors. The electrical signals created by the light detectors in response to reflected laser light 120, are communicated to the computing device (e.g. Computing system 110).

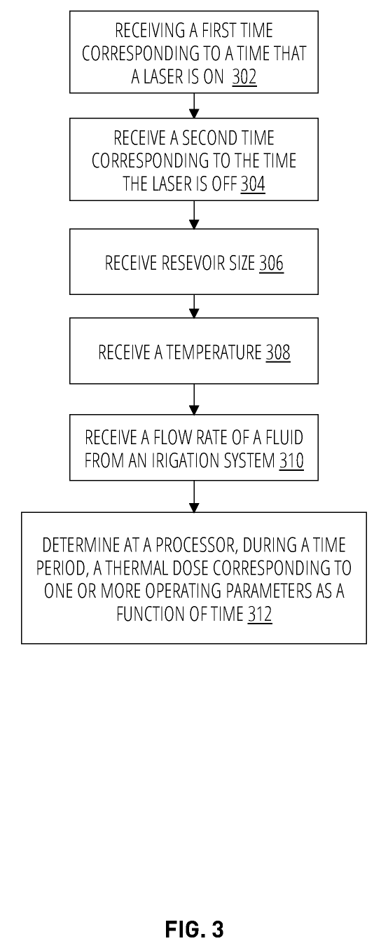

FIG. 3 illustrates a computer implemented method for calculating a thermal dose. The computer implemented method begins at block 302, “receiving a first time corresponding to a time that a laser is on". An electrical signal can be received by computing system 110. The electrical signal comprising an indication of how much time the laser was on or emitting laser light 116. The first time can be communicated to the computing system by the laser console or may be a predetermined duty cycle. The processor may execute instructions to receive an electrical signal indicating the first time.

Turning now to block 304 “receive a second time corresponding to the time that the laser is off". An electrical signal can be received, wherein the electrical signal comprises an indication of how much time the laser was off. The second time can be communicated to the computing system by the laser console. In some embodiments, the time that the laser is off is predetermined, and in some embodiments, it is manually adjusted throughout the procedure (e.g. duty cycle gating with a foot pedal). The processor may execute instructions to receive an electrical signal indicating the second time.

Continuing to block 306 “receiving a reservoir size”. The processor may execute instructions to receive an electrical signal corresponding to the size of the reservoir (e.g. kidney). The size of the reservoir may be input manually, calculated by the irrigation system, or determined by any number of known methods.

Continuing to block 308, “receiving a temperature”, the temperature can be determined by sensors, the irrigation system 128, or any number of known methods. In some embodiments, the temperature is predicted by a simulation calculation or a machine learning model configured to predict the temperature of the environment based on a variety of inputs. These inputs may include laser power, laser frequency, lasing on and off times, flow rate, reservoir volume, fluid temperature etc. In some embodiments, the temperature may be measured by a temperature sensor within the reservoir. The processor may execute instructions to receive an electrical signal corresponding to the predicted temperature and/or measured temperature.

Turning now to block 310, “receiving a flow rate and/or suction of a fluid from an irrigation system”. An irrigation system can be configured to monitor the rate of fluid flowing in and out of environment 124. The processor may execute instructions to receive an electrical signal corresponding to the flow rate and/or suction rate.

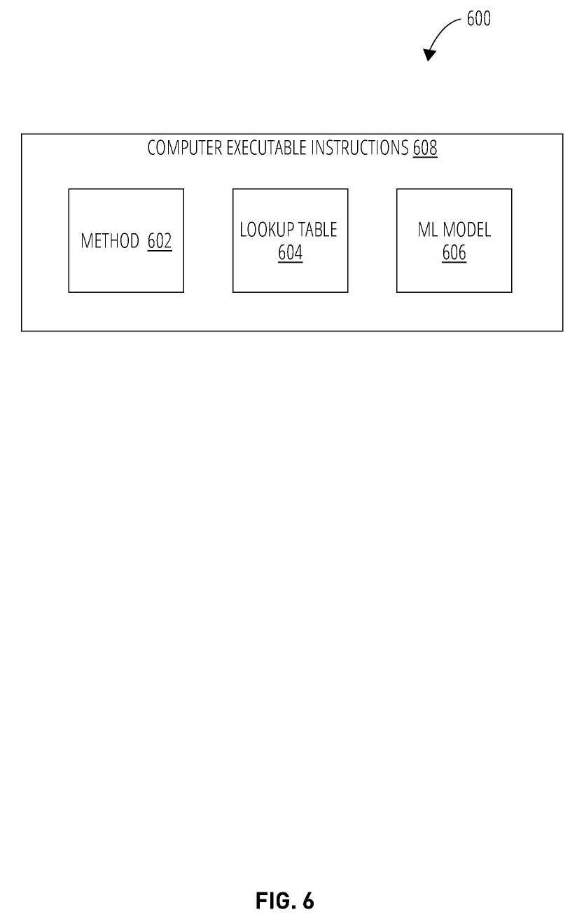

The method further includes block 312 “determining at a processor during a time period, a thermal dose corresponding to one or more operating parameters as a function of time”. The thermal dose can be calculated based on the electrical signals received in blocks 302, 304, 306, 308, and 310, over a time period. For example, memory storage device 706 can comprise a lookup table (FIG. 6) correlating operating parameters to a thermal dose. In yet another example, memory storage device 706 can comprise a trained machine learning (ML) model configured to receive as input, a plurality of operating parameters, and infer the thermal dose. In such an example, processor 704 can be configured to execute application instructions 724 to execute the ML to infer the thermal dose based on the determined operating parameters. As the thermal dose increases, the risk of tissue damage increases. If the thermal dose increases above a predetermined threshold, the procedure is not safe, and lasing should be stopped, or lasing frequency should be decreased. If the thermal dose is below a predetermined threshold, lasing can continue, and lasing frequency may be increased.

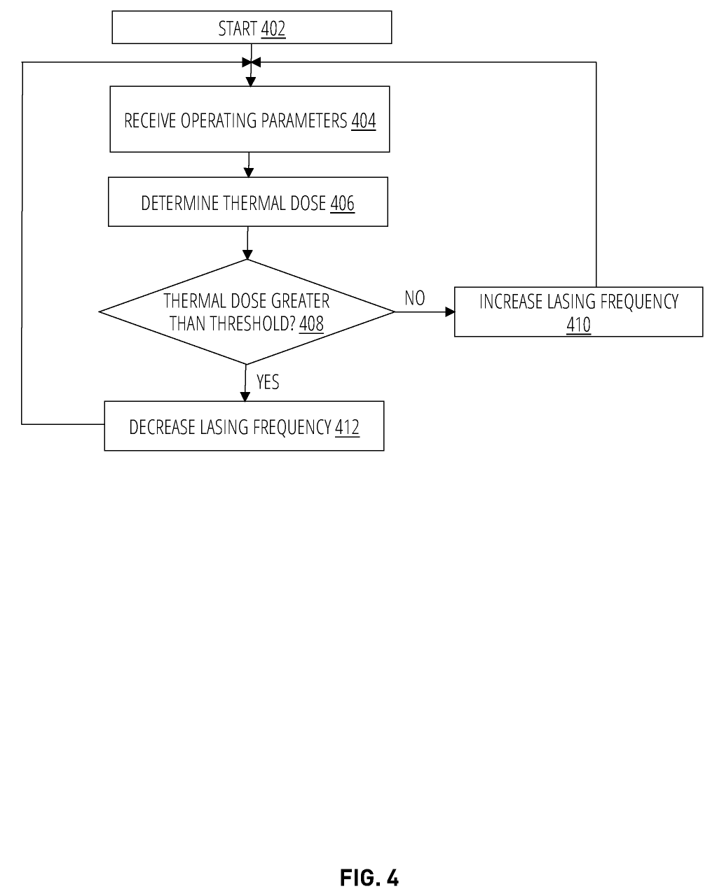

FIG. 4 illustrates a method for determining if the thermal dose is at a safe level. Operating parameters are received in block 404, (e.g. Duty cycle, temperature etc.). The thermal dose is calculated 406 and is then compared to a predetermined threshold amount at decision block 408. For example, processor 704 an execute application instructions 824 to determine whether the thermal dose determined at block 406 is greater than threshold. If the thermal dose is greater than threshold, the lasing frequency is decreased, and the method continues to decision block 412. In some embodiments, the lasing may be paused for a predetermined length of time or may be stopped until the thermal dose decreases below the predetermined threshold. If the thermal dose is below threshold, the lasing frequency is increased, and the method continues to decision block 410.

In some embodiments, distance measurements and/or target detection may also be included as an operating parameter, for example, to prevent adding to the thermal load of the temperature of the environment 124 when laser energy will not effectively reach a target. In general, thermal energy (or thermal dose) should only be delivered when a target is in range and targeted (e.g., aimed at, or the like), to increase efficiency and safety throughout the lithotripsy procedure. For example, when a target distance (e.g. stone, tumor, cyst etc.) is within a predetermined range, and the thermal dose is less than threshold, the lasing frequency is increased. In some embodiments, when the thermal dose is less than threshold, but the target distance is not within a predetermined range, lasing frequency will not be increased. As discussed in more detail above, the circuitry may be configured to determine when a stone is passing through the path of the laser light 116. In some embodiments, lasing frequency will be increased when a stone is passing through the path of the light, if the thermal dose is below threshold. Said differently, when the thermal dose is greater than threshold, lasing frequency will not be increased, regardless of the position of a stone or target.

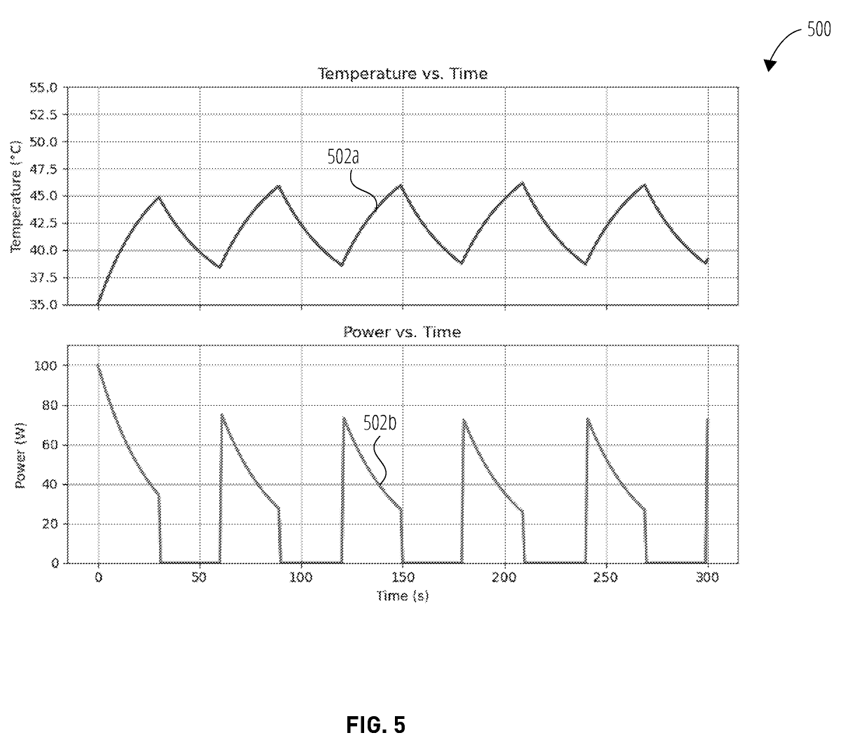

In general, the computing system 110 can include circuitry configured to receive multiple temperature parameters (e.g. duty cycle information, target or tissue location, irrigation fluid flow rate, irrigation fluid temperature, reservoir size, and the like) from the lasing system, fluid management system, or other inputs. The computing system can be configured to calculate an average power, a moving average power (e.g., average power over a rolling time period, or the like) or thermal dose corresponding to one or more of the temperature parameters over a period of time. As illustrated in FIG. 5, the computing system 110 is configured to calculate the thermal dose over a moving window. For example, continuously computing an average thermal dose over a predetermined time period throughout a lithotripsy procedure. In some embodiments, the computing system 110 is configured to graph the temperature over a period of time. The computing system may use a local temperature, a predicted temperature, or a calculated temperature. In some embodiments the computing system 110 is configured to graph power over time. The power may be a power of laser light delivered to the environment 124, or the power may be calculated using a combination of operating parameters, as discussed in more detail above.

FIG. 6 illustrates computer-readable storage medium 600. Computer-readable storage medium 614 may comprise any non-transitory computer-readable storage medium or machine-readable storage medium, such as an optical, magnetic or semiconductor storage medium. In various embodiments, computer-readable storage medium 614 may comprise an article of manufacture. In some embodiments, 614 may store computer executable instructions 610 with which circuitry (e.g., computing system 110, processor, or the like) can execute. For example, computer executable instructions 610 can include instructions to implement operations described with respect to method 602, computer executable instructions 608, lookup table 604, or ML model 606. Examples of computer-readable storage medium 614 or machine-readable storage medium may include any tangible media capable of storing electronic data, including volatile memory or non-volatile memory, removable or non-removable memory, erasable or non-erasable memory, writeable or re-writeable memory, and so forth. Examples of computer executable instructions 608 may include any suitable type of code, such as source code, compiled code, interpreted code, executable code, static code, dynamic code, object-oriented code, visual code, and the like.

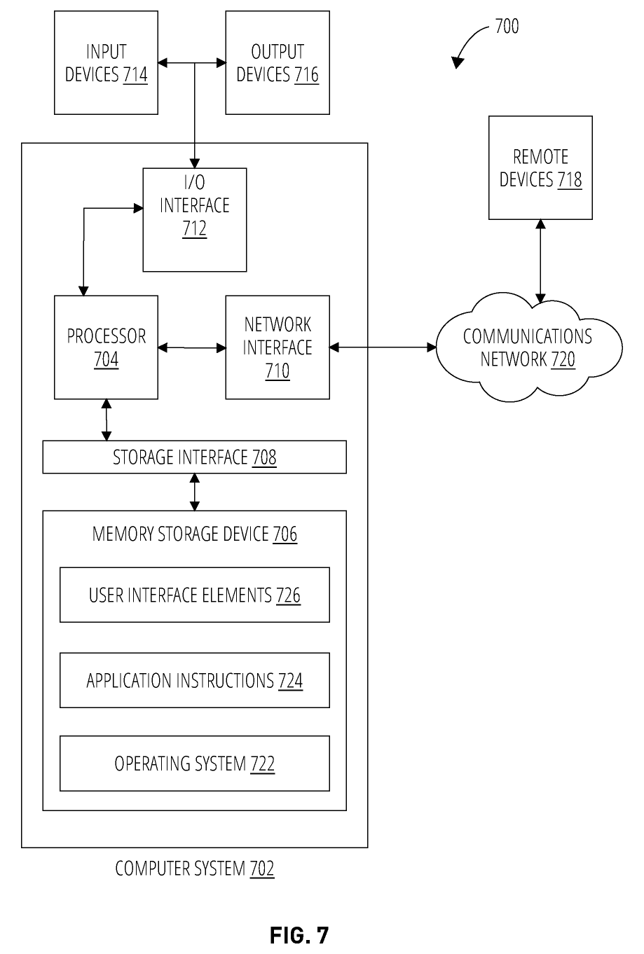

FIG. 7 is a block diagram of a computing environment 700 including a computer system 702 for implementing embodiments consistent with the present disclosure. In some embodiments, the computing environment 700, or portion thereof (e.g., the computer system 702) may comprise or be comprised in a laser system (e.g., the computing system 110 of the lithotripsy procedure system 100 can embody portions of the computing environment 700). Accordingly, in various embodiments, computer system 702 may determine a power of laser light emitted from a distal end of an optical fiber based on a duration of existence of a bubble formed by the laser light.

The computer system 702 may include a central processing unit (“CPU” or “processor”) 704. The processor 704 may include at least one data processor for executing instructions and/or program components for executing user or system-generated processes. A user may include a person, a person using a device such as those included in this disclosure, or another device. The processor 704 may include specialized processing units such as integrated system (bus) controllers, memory management control units, floating point units, graphics processing units, neural processing units, digital signal processing units, etc. The processor 704 may be disposed in communication with input devices 714 and output devices 716 via I/O interface 712. The I/O interface 712 may employ communication protocols/methods such as, without limitation, audio, analog, digital, stereo, IEEE-1394, serial bus, Universal Serial Bus (USB), infrared, PS/2, BNC, coaxial, component, composite, Digital Visual Interface (DVI), high-definition multimedia interface (HDMI), Radio Frequency (RF) antennas, S-Video, Video Graphics Array (VGA), IEEE 802.n /b/g/n/x, Bluetooth, cellular (e.g., Code-Division Multiple Access (CDMA), High-Speed Packet Access (HSPA+), Global System For Mobile Communications (GSM), Long-Term Evolution (LTE), or the like), etc.

Using the I/O interface 712, computer system 702 may communicate with input devices 714 and output devices 716. In some embodiments, the processor 704 may be disposed in communication with a communications network 720 via a network interface 710. In various embodiments, the communications network 720 may be utilized to communicate with a remote memory storage device 706, such as for accessing look-up tables, performing updates, or utilizing external resources. The network interface 710 may communicate with the communications network 720. The network interface 710 may employ connection protocols including, without limitation, direct connect, Ethernet (e.g., twisted pair 10/100/1000 Base T), Transmission Control Protocol/Internet Protocol (TCP/IP), token ring, IEEE 802.11a/b/g/n/x, etc.

The communications network 720 can be implemented as one of the different types of networks, such as intranet or Local Area Network (LAN), Closed Area Network (CAN) and such. The communications network 826 may either be a dedicated network or a shared network, which represents an association of the different types of networks that use a variety of protocols, for example, Hypertext Transfer Protocol (HTTP), CAN Protocol, Transmission Control Protocol/Internet Protocol (TCP/IP), Wireless Application Protocol (WAP), etc., to communicate with each other. Further, the communications network 720 may include a variety of network devices, including routers, bridges, servers, computing devices, storage devices, etcetera. In some embodiments, the processor 704 may be disposed in communication with a memory storage device 706 via a storage interface 708. The storage interface 708 may connect to memory storage device 706 including, without limitation, memory drives, removable disc drives, etc., employing connection protocols such as Serial Advanced Technology Attachment (SATA), Integrated Drive Electronics (IDE), IEEE-1394, Universal Serial Bus (USB), fiber channel, Small Computer Systems Interface (SCSI), etc. The memory drives may further include a drum, magnetic disc drive, magneto-optical drive, optical drive, Redundant Array of Independent Discs (RAID), solid-state memory devices, solid-state drives, etcetera.

Furthermore, memory storage device 706 may include one or more computer-readable storage media utilized in implementing embodiments consistent with the present disclosure. Generally, a computer-readable storage medium refers to any type of physical memory on which information or data readable by a processor may be stored. Thus, a computer-readable storage medium may store instructions for execution by one or more processors, including instructions for causing the processor(s) to perform steps or stages consistent with the embodiments described herein. The term “computer-readable medium” should be understood to include tangible items and exclude carrier waves and transient signals, i.e., non-transitory. Examples include Random Access Memory (RAM), Read-Only Memory (ROM), volatile memory, non-volatile memory, hard drives, Compact Disc (CD) ROMs, Digital Video Disc (DVDs), flash drives, disks, and any other known physical storage media.

The memory storage device 706 may store a collection of program or database components, including, without limitation, an operating system 722, application instructions 724, and user interface elements 726. In various embodiments, the operating system 722 may facilitate resource management and operation of the computer system 702. Examples of operating systems include, without limitation, APPLE^{®} MACINTOSH^{®} OS X^{®}, UNIX^{®}, UNIX-like system distributions (E.G., BERKELEY SOFTWARE DISTRIBUTION^{®} (BSD), FREEBSD^{®}, NETBSD^{®}, OPENBSD®, etc.), LINUX^{®} DISTRIBUTIONS (E.G., RED HAT^{®}, UBUNTU^{®}, KUBUNTU^{®}, etc.), IBM^{®}OS/2^{®}, MICROSOFT^{®} WINDOWS^{®} (XP^{®}, VISTA^{®}/7/8, 10 etc.), APPLE^{®} IOS^{®}, GOOGLE^{TM} ANDROID^{TM}, BLACKBERRY^{®} OS, or the like.

The application instructions 724 may include instructions that when executed by the processor 704 cause the processor 704 to perform one or more techniques, steps, procedures, and/or methods described herein, such as to determine a distance of a target disposed in a liquid environment (e.g., environment 124) based on a plurality of light signals (e.g., signal detector 208 and/or reference detector 206) formed by the laser light.

The user interface elements 726 may facilitate display, execution, interaction, manipulation, or operation of program components through textual or graphical facilities. For example, user interfaces may provide computer interaction interface elements on a display system operatively connected to the computer system 702, such as cursors, icons, checkboxes, menus, scrollers, windows, widgets, etcetera. The user interface elements 726 may be employed by application instructions 724 and/or operating system 722 to provide, for example, a user interface with which a user can interact with computer system 702. In some embodiments, the user interface elements 726 may be displayed on a display. With some embodiments, the user interface elements 726 can comprise auditory (adjustment of pinging to reflect changes in laser activation parameters) and/or tactile or haptic feedback (e.g., via the endoscope handle, or the like).

Herein, references to "one embodiment" or "an embodiment" do not necessarily refer to the same embodiment, although they may. Unless the context clearly requires otherwise, throughout the description and the claims, the words "comprise," "comprising," and the like are to be construed in an inclusive sense as opposed to an exclusive or exhaustive sense; that is to say, in the sense of "including, but not limited to." Words using the singular or plural number also include the plural or singular number respectively, unless expressly limited to one or multiple ones. Additionally, the words "herein," "above," "below" and words of similar import, when used in this application, refer to this application as a whole and not to any portions of this application. When the claims use the word "or" in reference to a list of two or more items, that word covers all the following interpretations of the word: any of the items in the list, all the items in the list and any combination of the items in the list, unless expressly limited to one or the other. Any terms not expressly defined herein have their conventional meaning as commonly understood by those having skill in the relevant art(s).

What is claimed is:

1. A medical laser console comprising:
a laser source configured to generate laser light;
an optical coupler to couple the laser light to an optical fiber; and
a controller, the controller comprising at least a processor and a memory, the memory comprising instructions, when executed by the processor, cause the controller to:
receive a plurality of operating parameters, including:
a first time generated by a laser system, the first time corresponding to a time that a laser is on, and
a second time generated by the laser system, the second time corresponding to a time that the laser is off;
determine a temperature; and
send a control signal to the laser source to cause the laser source to adjust one or more of the operating parameters based on the temperature.

2. The medical laser console of claim 1, wherein the instructions, when executed by the processor cause the controller to:
determine, during a time period, a thermal dose corresponding to the operating parameters and the temperature,
wherein the thermal dose is represented as a function of time.

3. The medical laser console of claim 2, further comprising an irrigation system.

4. The medical laser system of claim 2, wherein the plurality of operating parameters further includes a fluid temperature from the irrigation system.

5. The medical laser console of claim 4, wherein the plurality of operating parameters further includes a fluid flow rate and/or suction rate from the irrigation system.

6. The medical laser console of claim 2, wherein the instructions, when executed by the processor cause the controller to:
identify a reference in a lookup table corresponding to a thermal dose, wherein the lookup table is stored in a memory coupled to the processor, and wherein the lookup table correlates the plurality of operating parameters to the thermal dose; and
determine the thermal dose based on the reference.

7. The medical laser console of claim 2, wherein the instructions, when executed by the processor cause the controller to:
determine whether the thermal dose is greater than or equal to a threshold thermal dose; and
send a control signal to the laser source to cause the laser source to stop generating the laser light responsive to a determination that the thermal dose is greater than or equal to the threshold thermal dose.

8. The medical laser console of claim 2, wherein the instructions, when executed by the processor cause the controller to:
compare the thermal dose to a predetermined temperature threshold; and
determine if the thermal dose is safe or unsafe,
wherein as the thermal dose increases above a predetermined threshold, the thermal dose becomes unsafe, and wherein as the thermal dose decreases below a predetermined threshold, the thermal dose becomes safe.

9. The medical laser console of claim 8, wherein the instructions, when executed by the processor, are configured to decrease a frequency of lasing when the thermal dose is unsafe and increase a frequency of lasing when the thermal dose is safe.

10. The medical laser console of claim 9, further comprising determining at the processor, a distance of one or more of a plurality of objects from a distal end of the optical fiber.

11. The medical laser console of claim 1, wherein the instructions, when executed by the processor, cause the controller to:
compare the distance to a predetermined threshold distance; and
determine if the one or more of a plurality of objects is in range, or out of range.

12. The medical laser console of claim 11, wherein the instructions, when executed by the processor, are configured to increase the frequency of lasing when the one or more of the plurality of objects is in range.

13. A method of determining a thermal dose, the method comprising:
receiving, at a controller for a surgical laser system, a plurality of operating parameters, including:
a first time generated by a laser system, the first time corresponding to a time that a laser is on;
a second time generated by the laser system, the second time corresponding to a time that the laser is off;
determining, using a simulation model, a temperature prediction based on the plurality of operating parameters; and
determining, during a time period, a thermal dose corresponding to the temperature prediction as a function of time.

14. The method of claim 13, further comprising:
comparing the thermal dose to a predetermined temperature threshold; and
determining if the thermal dose is safe or unsafe.

15. The method of claim 14, wherein as the thermal does increases above a predetermined threshold, the thermal dose becomes unsafe, and wherein as the thermal dose decreases below a predetermined threshold, the thermal dose becomes safe.

16. The method of claim 14, further comprising decreasing a frequency of lasing when the thermal dose is unsafe and increase a frequency of lasing when the thermal dose is safe.

17. At least one non-transitory computer-readable storage medium comprising instructions executable by a processor of a surgical laser console, which instructions, when executed, cause the surgical laser console to:
receive a plurality of operating parameters, including:
a first time generated by a laser system, the first time corresponding to a time that a laser source of the surgical laser console is on;
a second time generated by the laser system, the second time corresponding to a time that the laser source is off; and
determine, using a model, a predicted temperature based on the plurality of operating parameters.

18. The at least one non-transitory computer-readable storage medium of claim 17, wherein the predicted temperature is determined based on a machine learning model.

19. The at least one non-transitory computer-readable storage medium of claim 17, wherein the predicted temperature is derived by an algorithm.

20. The at least one non-transitory computer-readable storage medium of claim 17, wherein the instructions when executed by the processor further cause the surgical laser system to:
determine whether the thermal dose is greater than or equal to a threshold thermal dose; and
send a control signal to the laser source to cause the laser source to stop generating the laser light responsive to a determination that the thermal dose is greater than or equal to the threshold thermal dose.
